(19) United States
(12) Patent Application Publication
(10) Pub. No.: US 2018/0237775 A1
(43) Pub. Date: Aug. 23, 2018

(54) ANTISENSE OLIGONUCLEOTIDES AND USES THEREOF

(71) Applicant: ASSOCIATION INSTITUT DE MYOLOGIE, Paris (FR)

(72) Inventors: Julie DUMONCEAUX, Ealing (GB); Thomas VOIT, London (GB); John George DICKSON, London (GB); Linda Jane POPPLEWELL, Berkshire (GB)

(21) Appl. No.: 15/761,804

(22) PCT Filed: Sep. 21, 2016
(86) PCT No.: PCT/EP2016/072447

§ 371 Date: Mar. 20, 2018

(30) Foreign Application Priority Data
Sep. 21, 2015 (EP) 15306467.0

(51) Int. Cl. C12N 15/113; C12N 15/86

(52) U.S. Cl. CPC C12N 15/113; C12N 15/86; C12N 2310/315; C12N 2310/3233; C12N 2310/321; C12N 2310/11

(57) ABSTRACT

The present invention relates to nucleic acids, compositions and methods for the treatment of diseases, in particular for the treatment of facioscapulohumeral dystrophy.

### FIELD OF THE INVENTION

The present invention relates to nucleic acids, compositions and methods for the treatment of diseases, in particular of facioscapulohumeral dystrophy.

### BACKGROUND OF THE INVENTION

The cleavage and polyadenylation of the 3′ end are fundamental processing steps for the maturation of the vast majority of eukaryotic mRNAs. In Human, these reactions are governed by more than 80 RNA-binding proteins and by regulatory cis-acting RNA sequence elements (for reviews see (Elkon, Ugalde et al. 2013)(Nunes, Li et al. 2010)). The key element dictating the cleavage is a 6 nucleotide (nt) motif called the poly(A) signal (PAS). Most of the mammalian mRNAs contain the consensus AAUAAA or AAUAAA hexamer or close variants (Tian, Hu et al. 2005)(Beaudoing, Freier et al. 2000) which is recognized by cleavage and polyadenylation factors. This RNA-protein interaction determines the site of cleavage which occurs 10-30 nt downstream the PAS. The second important element is a U/GU-rich sequence contacted by the cleavage stimulation factor (CstF) and located 30-45 nt downstream the PAS motif (for review see (Nunes, Li et al. 2010)). In most cases, these co-transcriptional maturations are required for nuclear export, stability of the mRNA and efficient translation (Sachs 1990) and consequently could represent interesting targets for suppression of gene expression. Indeed, the functional importance of the 3′ end mRNA processing has been highlighted by the discovery of mutations in the PAS cis-element causing or contributing to human diseases including thalassemias in whom the alteration of the AAUAAA hexanucleotide leads to a loss of function of globin 3′ end processing inactivating or severely inhibiting α- or β-globin gene expression (for reviews see (Danckwardt, Hentze et al. 2008)(Elkon, Ugalde et al. 2013)).

Targeting PAS using antisense oligonucleotides for gene silencing has never been proposed in the prior art. We focused on FacioScapuloHumeral Dystrophy (FSHD) which is a rare autosomal dominant neuromuscular disorder with an incidence of 1:14,000 to 1:20,000 (Tawil, van der Maarel et al. 2014). This pathology is caused by a loss of epigenetic marks within the D4Z4 macrosatellite located in the sub-telomeric region of chromosome 4 leading to chromatin relaxation (van der Maarel, Miller et al. 2012). In 95% of the FSHD patients (named FSHD1), this chromatin relaxation is associated with a contraction of the D4Z4 array (van Deutekom, Wijmenga et al. 1993)(Wijmenga, Hewitt et al. 1992) whereas the remaining 5% of the FSHD patients do not present a contraction of D4Z4 but the vast majority of them carry a mutation in the epigenetic modifier gene SMCHD1 (Lemmers, Tawil et al. 2012)(Lemmers, Goeman et al. 2014). This loss of epigenetic marks, when associated with a permissive chromosome 4, leads to the aberrant transcription of a double homeobox transcription factor named DUX4 whose ORF is present in each D4Z4 repeat (Gabriels, Beckers et al. 1999)(Snider, Asawachaicharn et al. 2009). DUX4 protein and mRNA have been robustly detected in adult and fetal FSHD1 and FSHD2 cells and biopsies whereas they were rarely found in control (Snider, Geng et al. 2010; Jones, Chen et al. 2012; Broucqsault, Morere et al. 2013; Ferreboeuf, Mariot et al. 2014). DUX4 is a transcription factor and its overexpression is described to disturb several cellular pathways (Kowaljow, Marcowycz et al. 2007)(Dixit, Ansseau et al. 2007; Vanderplanck, Ansseau et al. 2011; Wallace, Garwick et al. 2011)(Geng, Yao et al. 2012)(Vanderplanck, Ansseau et al. 2011)(Xu, Wang et al. 2014)(Wallace, Garwick et al. 2011). Moreover, it was recently shown that even if DUX4 expression has not been directly linked to patient's phenotype, DUX4 may play a major role in the pathophysiology of FSHD because: (i) it has been shown that at least one D4Z4 repeat is needed for FSHD onset (Tupler, Berardinelli et al. 1996), (ii) only alleles with the 4qA type (containing the AUUAAA PAS for DUX4 mRNA) are associated with FSHD patients (Lemmers, de Kievit et al. 2002; Thomas, Wiseman et al. 2007), (iii) contraction of the D4Z4 array on chromosome 10 which carries a mutated PAS (AUCAAA) does not lead to FSHD, (iv) DUX4-induced gene expression is the major molecular signature in FSHD skeletal muscles (Yao, Snider et al. 2014), and (v) DUX4 expression is the common point between FSHD1 and FSHD2 patients (Lemmers, van der Vliet et al. 2010).

Several therapeutic strategies targeting DUX4 expression have been proposed in the literature: RNA interference, 2′-O-methyl antisense oligonucleotides (AO) targeting intron-exon junctions or overexpression of truncated DUX4 (Vanderplanck, Ansseau et al. 2011; Geng, Yao et al. 2012; Wallace, Liu et al. 2012; Mitsuhashi, Mitsuhashi et al. 2013). However, there still remains a need for efficient or alternative therapeutic strategies of FSHD.

### SUMMARY OF THE INVENTION

Here we describe a new therapeutic antisense oligonucleotide (AO)-based approach for the treatment of genetic diseases, in particular FSHD, targeting the key elements of 3′ end processing of a pre-mRNA.

Accordingly, the present invention relates to an antisense oligonucleotide that hybridizes with at least one key element of the polyadenylation region of a target pre-mRNA.

The invention further relates to an antisense oligonucleotide that hybridizes with the key elements of 3′ end processing of a target pre-mRNA, in particular the polyadenylation signal, the cleavage site(s) and/or the U/GU-rich region of the polyadenylation region of a target pre-mRNA, for use in a method for the treatment of a disease mediated by said target pre-mRNA or protein encoded by said pre-mRNA.

In a further aspect, the invention relates to a method for the treatment of a disease, comprising administering to a subject in need thereof an antisense oligonucleotide that hybridizes with at least one key element of 3′ end processing of a pre-mRNA, such as the polyadenylation region of a target pre-mRNA, wherein said disease is mediated by said target pre-mRNA or protein encoded by said pre-mRNA.

### DETAILED DESCRIPTION OF THE INVENTION

The present invention relates to an antisense oligonucleotide useful for treating a subject suffering from a disease resulting from the abnormal expression of a protein. In a specific embodiment, the subject has FSHD and the pre-mRNA targeted by the antisense oligonucleotide is a DUX4 pre-mRNA.

In the present application, “antisense oligonucleotide”, or “AO” denotes a single stranded nucleic acid sequence, either DNA or RNA (Chan et al., 2006), which is complementary to a part of a pre-mRNA coding a protein which is abnormally expressed in a cell, such as the pre-mRNA coding DUX4 in a FSHD patient. Specifically, the AO of the present invention is designed to hybridize with the targeted pre-mRNA at a location comprising key elements of 3′ pre-mRNA processing such as the polyadenylation site, cleavage site(s) and the U:GU-rich region (or DSE for DownStream Element) of said pre-mRNA.

The AO is used for silencing the expression of a target protein which is abnormally expressed in a cell or tissue. Without wishing to be bound by any theory, we believe that the proposed AO strategy prevents the correct maturation of said pre-mRNA to occur, either preventing its cleavage or its polyadenylation, for example. Being not correctly processed, or not fully correctly processed, the targeted pre-mRNA is not further translated into the encoded protein. Although the data presented below are focused on the treatment of FSHD with AOs targeting key elements of 3′ DUX4 pre-mRNA processing, it is anticipated that the mechanisms underlying the observed results are applicable as well to other pre-mRNAs and diseases.

The AO of the invention is designed to complement suitable sequences within the target pre-mRNA, which are required for correct polyadenylation event, thereby blocking its maturation. The AO of the invention targets at least one, or all, the key elements required for correct polyadenylation, such as the polyadenylation signal, cleavage site(s) and the U/Gu-rich region of the polyadenylation region of a given pre-mRNA. These elements are well-known to those skilled in the art (such as in Nunes, Li et al., 2010 and Hollerer, Grund et al., 2014)

AOs employed in the practice of the invention are generally from about 10 to about 40 nucleotides in length, and may be for example, about 10, or about 15, or about 20, or about 25, or about 30, or about 35, or about 40 nucleotides or more in length depending on the targeted sequences within the target pre-mRNA, in particular the target DUX4 pre-mRNA and the AO chemistry.

The AO of the invention is able to hybridize to the target sequence with high or severe stringency. Severe or high stringency conditions comprise, for example, overnight hybridization at about 68° C. in a 6×SSC solution followed by washing at about 68° C. in a 0.6×SSC solution. As such the present invention relates to an AO which is able to silence the expression of a target protein, and which is at least 80%, at least 85%, at least 90%, at least 95%, or event at least 96%, at least 97%, at least 98% or at least 99% to the targeted region of the targeted pre-mRNA encoding said target protein. In a further embodiment, the AO may comprise a gap when compared to the targeted region of the target pre-mRNA. In a preferred embodiment, the AO of the invention is 100% complementary to the targeted region of the target pre-mRNA.

In a particular embodiment, the AO is designed to hybridize with the targeted pre-mRNA at or about the polyadenylation region of said target pre-mRNA. In a particular embodiment, the AO complements with a target sequence within the targeted pre-mRNA including the polyadenylation signal, and spanning 5′ and/or 3′ from said polyadenylation signal. In the following, the +1 nucleic acid is the first nucleotide of the polyadenylation signal; nucleotides 5′ from this polyadenylation signal are negatively numbered (for example, the third nucleotide 5′ from the polyadenylation signal is numbered −3); nucleotides 3′ from the polyadenylation signal are positively numbered (for example, the fifth nucleotide from the first nucleotide of the polyadenylation signal (and including the latter) is numbered +5). In particular variants of this embodiment, the AO of the invention targets a nucleic acid sequence which is included in the (−20 +20) region of the pre-mRNA. In another embodiment, the targeted sequence is within the (−10 +30) region of the targeted pre-mRNA.

In another embodiment, the targeted region includes all or a part of the polyadenylation signal. For example, the AO may target a sequence whose most 5′ nucleotide within the pre-RNA is the +2, +3 or +4 nucleotide (in relation to the first nucleotide of the polyadenylation signal).

In a further particular embodiment of the invention, the targeted region does not include the polyadenylation signal, but includes one or more key elements required for polyadenylation such as cleavage site(s) and/or the U/GU-rich region. These regions in a given pre-mRNA are well-known in the art, and may be readily determined to those skilled in the art. Examples of such AO targeting cleavage site(s) or the U/GU-rich region are provided in the experimental part below, were AOs specific to the DUX4 pre-mRNA are presented.

In a particular embodiment, the targeted pre-mRNA is a DUX4 pre-mRNA. In specific embodiments of the invention, the AO targeting a DUX4 pre-mRNA is one selected from those listed in table 1:

| Targeted | | |
|---|---|---|
| element | Sequence | SEQ ID |
| polyA | 5′ GGGCATTTTAATATA | SEQ ID NO: 1 |
| | TCTCTGAACT 3′ | |
| CS1 | 5′ CTATAGGATCCACAG | SEQ ID NO: 2 |
| | GGCATTTTAATATC 3′ | |
| CS2 | 5′ GGATCCACAGGGAGG | SEQ ID NO: 3 |
| | AGGCATTTTAATA 3′ | |
| CS3 | 5′ TATAGGATCCACAGG | SEQ ID NO: 4 |
| | GAGGAGGCATTTTAA 3′ | |
| DSE | 5′ CATCACACAAAAGAT | SEQ ID NO: 5 |
| | GCAAATCTTC 3′ | |

The AO of the invention may be of any suitable type. Representative AO types include oligodeoxyribonucleotides, oligoribonucleotides, morpholinos, tricyclo-DNA-antisense oligonucleotides, tricyclo-phosphorothioate DNA oligonucleotides, LNA, small nuclear RNA-modified such as U7-, U1- or U6-modified AOs (or other UsnRNPs), or conjugate products thereof such as peptide-conjugated or nanoparticle-complexed AOs.

For use in vivo, the AO may be stabilized, for example via phosphate backbone modifications. For example, stabilized AOs of the instant invention may have a modified backbone, e.g. have phosphorothioate linkages. Other possible stabilizing modifications include phosphodiester modifications, combinations of phosphodiester and phosphorothioate modifications, methylphosphonate, methylphosphorothioate, phosphorodithioate, p-ethoxy, and combinations thereof. Chemically stabilized, modified versions of the AOs also include “Morpholinos” (phosphorodiamidate morpholino oligomers, PMOs), 2′-O-Methyl oligomers, tricyclo-DNAs, tricyclo-DNA-phosphorothioate AON molecules (WO2013/053928) or U small nuclear (sn) RNAs. The latter forms of AOs that may be used to this effect can be coupled to small nuclear RNA molecules such as U1, U6 or U7 (or other UsnRNPs), in particular in combination with a viral transfer method based on, but not limited to, lentivirus, retrovirus or adeno-associated virus.

In a specific embodiment of the invention, the AO of the invention is a PMO AO.

In a particular embodiment, the AO of the invention, more particularly a PMO AO, more particularly a PMO AO which is an uncharged oligonucleotide, is annealed to a sense oligonucleotide, or a so-called “leash” to facilitate AO entry into cells. In a particular embodiment, the leash is designed so that its hybridization with the AO results in unpaired protruding nucleotides at both the 5′ and 3′ ends of the leash. According to a particular embodiment, as a result of hybridization of the AO and the leash, the AO is partly annealed to the leash, thereby providing the AO with either a 5′-, 3′- or both 5′- and 3′-protruding ends. In a particular embodiment, the AO is a PMO AO and the leash is a DNA oligonucleotide. In a particular embodiment, the leash hybridizes with 15-18 nucleotides of AO of the invention. In a particular embodiment, protruding 5′ and 3′ ends comprise, independently one from the other, 1, 2, 3, 4, 5 or more than 5 unpaired nucleotides. Such leashes are shown in FIG. 1 for AOs that are specific to the DUX4 pre-mRNA.

Antisense sequences of the invention may be delivered in vivo alone or in association with a vector. In its broadest sense, a “vector” is any vehicle capable of facilitating the transfer of the antisense sequence to the cells and preferably cells expressing DUX4. Preferably, the vector transports the antisense sequence to cells with reduced degradation relative to the extent of degradation that would result in the absence of the vector. In general, the vectors useful in the invention include, but are not limited to, plasmids, phagemids, viruses, and other vehicles derived from viral or bacterial sources that have been manipulated by the insertion or incorporation of the AO sequence. Viral vectors are a preferred type of vector and include, but are not limited to, nucleic acid sequences from the following viruses: lentivirus such as HIV-1, retrovirus, such as moloney murine leukemia virus, adenovirus, adeno-associated virus (AAV); SV40-type viruses; Herpes viruses such as HSV-1 and vaccinia virus. One can readily employ other vectors not named but known in the art. Among the vectors that have been validated for clinical applications and that can be used to deliver the antisense sequences, lentivirus, retrovirus and AAV show a greater potential and are preferred viral vectors of the invention. In a particular embodiment of the invention, the target cell is a cell of the muscular lineage, such as a myoblast, or a myotube, or a mature myofibre. In a further embodiment, the vector used for targeting said cell of the muscular lineage is a lentivirus or an AAV.

In a particular embodiment, the viral vector is an AAV vector. The serotype of the AAV vector is selected by one skilled in the art depending on the target cell that must be transduced by said AAV vector. In a particular embodiment, the target cell is of the muscle lineage, and the capsid of the AAV vector is from serotype 1, 6, 8 or 9 of AAV. In a further particular embodiment, the AAV vector is a pseudotyped vector, i.e. its genome and capsid are derived from AAVs of different serotypes. For example, the pseudotyped AAV vector may be a vector whose genome is derived from the AAV2 serotype, and whose capsid is derived from the AAV1, 3, 4, 5, 6, 7, 8, 9, 10 (e.g. cynomolgus AAV10 or AAVrh10), 11, 12 serotype or from AAV variants. In a particular embodiment, the AAV vector is pseudotyped and the AAV capsid is derived from the AAV1, 6, 8 or 9 serotype. In addition, the genome of the AAV vector may either be a single stranded or self-complementary double-stranded genome (McCarty et al., 2001). Self-complementary double-stranded AAV vectors are generated by deleting the terminal resolution site (trs) from one of the AAV terminal repeats. These modified vectors, whose replicating genome is half the length of the wild type AAV genome have the tendency to package DNA dimers.

In a particular embodiment, the AO as described above is linked to a small nuclear RNA molecule such as a U1, U2, U6, U7 or any other small nuclear RNA, or chimeric small nuclear RNA (Cazzella et al., 2012; De Angelis et al., 2002). Information on U7 modification can in particular be found in Goyenvalle, et al. (Goyenvalle et al., 2004); WO11113889; and WO06021724. In a particular embodiment, the U7 cassette described by D. Schumperli is used (Schumperli and Pillai, 2004). It comprises the natural U7-promoter (position −267 to +1), the U7smOpt snRNA and the downstream sequence down to position 116. The 18 nt natural sequence complementary to histone pre-mRNAs in U7smOpt is replaced by the selected AO sequence using, for example, PCR-mediated mutagenesis, as already described (Goyenvalle et al., 2004).

In a particular embodiment, the small nuclear RNA-modified AO, in particular the U7-modified AO, is vectorized in a viral vector, more particularly in a retroviral, lentiviral or AAV vector.

Typically, the vector may also comprise regulatory sequences allowing expression of the encoded AOs, such as e.g., a promoter, enhancer internal ribosome entry sites (IRES), sequences encoding protein transduction domains (PTD), and the like. In this regard, the vector most preferably comprises a promoter region, operably linked to the coding sequence, to cause or improve expression of the AO. Such a promoter may be ubiquitous, tissue-specific, strong, weak, regulated, chimeric, etc., to allow efficient and suitable production of the AON. The promoter may be a cellular, viral, fungal, plant or synthetic promoter. Most preferred promoters for use in the present invention shall be functional in muscle cells. Promoters may be selected from small nuclear RNA promoters such as U1, U2, U6, U7 or other small nuclear RNA promoters, or chimeric small nuclear RNA promoters. Other representative promoters include RNA polymerase III-dependent promoters, such as the H1 promoter, or RNA polymerase II-dependent promoters. Examples of regulated promoters include, without limitation, Tet on/off element-containing promoters, rapamycin-inducible promoters and metallothionein promoters. Examples of promoters specific for muscle cells include the C512 and desmin promoter. Examples of ubiquitous promoters include viral promoters, particularly the CMV promoter, the RSV promoter, the SV40 promoter, hybrid CBA (Chicken beta actin/CMV) promoter, etc. and cellular promoters such as the PGK (phosphoglycerate kinase) or EF1alpha (Elongation Factor 1 alpha) promoters.

In a particular embodiment, the AO used in the present invention is vectorized in a viral vector, in particular a retroviral, lentiviral or AAV vector, and comprises, for example, one or more of the sequences shown in SEQ ID NO:1-5. In addition, in a further particular embodiment, the vectorized AO comprises a small nuclear molecule such as U1, U6 or U7 (or other UsnRNPs), in particular U7.

The invention also relates to a composition comprising the AO of the invention, either alone or annealed to a leash as described above, or comprising a vector comprising an antisense oligonucleotide as described above. In addition to the AO or to the vector, a pharmaceutical composition of the present invention may also include a pharmaceutically or physiologically acceptable carrier such as saline, sodium phosphate, etc. The composition will generally be in the form of a liquid, although this needs not always to be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, cellulose, water syrup, methyl cellulose, methyl and propylhydroxybenzoates, mineral oil, etc. The formulation can also include lubricating agents, wetting agents, emulsifying agents, preservatives, buffering agents, etc. In particular, the present invention involves the administration of an AO or of a vector, such as a viral vector, and is thus somewhat akin to gene therapy. Those of skill in the art will recognize that nucleic acids are often delivered in conjunction with lipids (e.g. cationic lipids or neutral lipids, or mixtures of these), frequently in the form of liposomes or other suitable micro- or nano-structured material (e.g. micelles, lipocomplexes, dendrimers, emulsions, cubic phases, etc.). Thus the present invention also relates to a composition comprising an AO as described above, optionally annealed to a leash as described above, and a nucleic acid transfection reagent, such as a cationic lipid transfection reagent such as Lipofectamine® RNAiMax Reagent (Life Technologies). The AO of the invention may also be fused to or co-administrated with any cell-penetrating peptide and to signal peptides mediating protein secretion. Cell-penetrating peptides can be RVG peptides (Kumar et al., 2007), PiP (Betts et al., 2012), P28 (Yamada et al., 2013), or protein transduction domains like TAT (Malhotra et al., 2013) or VP22 (Lundberg et al., 2003)

The compositions of the invention are generally administered via enteral or parenteral routes, e.g. intravenously (i.v.), intra-arterially, subcutaneously, intramuscularly (i.m.), intracerebrally, intracerebroventricularly (i.c.v.), intrathecally (i.t.), intraperitoneally (i.p.), although other types of administration are not precluded, e.g. via inhalation, intranasally, topical, per os, rectally, intraosseous, eye drops, ear drops administration, etc.

Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol. While delivery may be either local (i.e. in situ, directly into tissue such as muscle tissue) or systemic, usually delivery will be local to affected muscle tissue, e.g. to skeletal muscle, smooth muscle, heart muscle, etc. Depending on the form of the AOs that are administered and the tissue or cell type that is targeted, techniques such as electroporation, sonoporation, a “gene gun” (delivering nucleic acid-coated gold particles), etc. may be employed.

One skilled in the art will recognize that the amount of an AO, or of a vector containing the AO, to be administered will be an amount that is sufficient to induce amelioration of unwanted disease symptoms (such as FSHD symptoms). Such an amount may vary inter alia depending on such factors as the gender, age, weight, overall physical condition of the patient, etc. and may be determined on a case by case basis. The amount may also vary according to other components of a treatment protocol (e.g. administration of other medicaments, etc.). Generally, a suitable dose is in the range of from about 1 mg/kg to about 100 mg/kg, and more usually from about 2 mg/kg/day to about 10 mg/kg. If a viral-based delivery of AON is chosen, suitable doses will depend on different factors such as the virus that is employed, the route of delivery (intramuscular, intravenous, intra-arterial or other), but may typically range from 10e9 to 10e15 viral particles/kg. Those of skill in the art will recognize that such parameters are normally worked out during clinical trials. Further, those of skill in the art will recognize that, while disease symptoms may be completely alleviated by the treatments described herein, this need not be the case. Even a partial or intermittent relief of symptoms may be of great benefit to the recipient. In addition, treatment of the patient may be a single event, or the patient is administered with the AO or the vector on multiple occasions, that may be, depending on the results obtained, several days apart, several weeks apart, or several months apart, or even several years apart.

Further aspects and advantages of the present inventions will be disclosed in the following experimental section, which shall be considered as illustrative only, and not limiting the scope of this application.

### LEGENDS OF THE FIGURES

FIG. 1: PMOs used in this study

A: sequences of the PMOs and leashes used. The bases in lower case do not match with the PMO sequence.

B: Positions of the different PMOs on DUX4 pre-mRNA. DUX4 polyA signal is indicated in bold. The dotted lines in PMO-CS1 corresponds to the deletions introduced in the PMO-CS1. The A in PMO-CS2 and -CS3 corresponds to point mutation introduced in these PMOs. Vertical double arrows correspond to cleavage sites identified within the DUX4 pre-mRNA. The exact positions of the PMOs are indicated and numbers correspond to the annealing coordinates. Position +1 is defined as the beginning of the polyA site.

FIG. 2: PMOs targeting the 3′ key elements of DUX4 mRNA induce a down-regulation of DUX4 FSHD cells were transfected with PMOs at different concentrations. Cells were harvested 4 days after induction of differentiation, and 2 days after transfection. Total RNAs were extracted and a reverse transcription using poly dT oligonucleotide (GCGAGCTCCGCGGCCGCGTTTTTTTTTTTVN; SEQ ID NO:6) was performed. DUX4 PCRs were performed and representative gels are shown in A. In B, the percentage of residual DUX4 mRNA is indicated and at least 3 experiments were analyzed.

FIG. 3: PMOs induces a down-regulation of several genes downstream of DUX4 FSHD cells were transfected with PMOs at different concentrations. Cells were harvested 4 days after induction of differentiation, and 2 days after transfection and expression levels of several genes downstream of DUX4 were measured by RT-qPCR. Results represent the mean of at least 4 experiments. B2M was used as the reference gene.

FIG. 4: PMO-CS3 induces a switch in cleavage site usage.

A redirection of poly(A) usage was investigated in the presence of the different PMOs. (A) 3′RACE nested PCR using forward primers located in Exon 3 shows a switch in cleavage site only in the presence of PMO-CS3. The bands with (double asterisks) or without (single asterisk) a redirection of the cleavage site are indicated. (B) The sequence of the most abundant mRNA carrying the redirected cleavage site (DUX4 pre-mRNA). The sequence of poly(A) site is underlined and bolded. The poly(A) tail is in bold. The frequencies of each variant showing alternative cleavage site usage are indicated (14 analyzed sequences).

### EXAMPLES

Material and Methods

PMO Design and Synthesis:

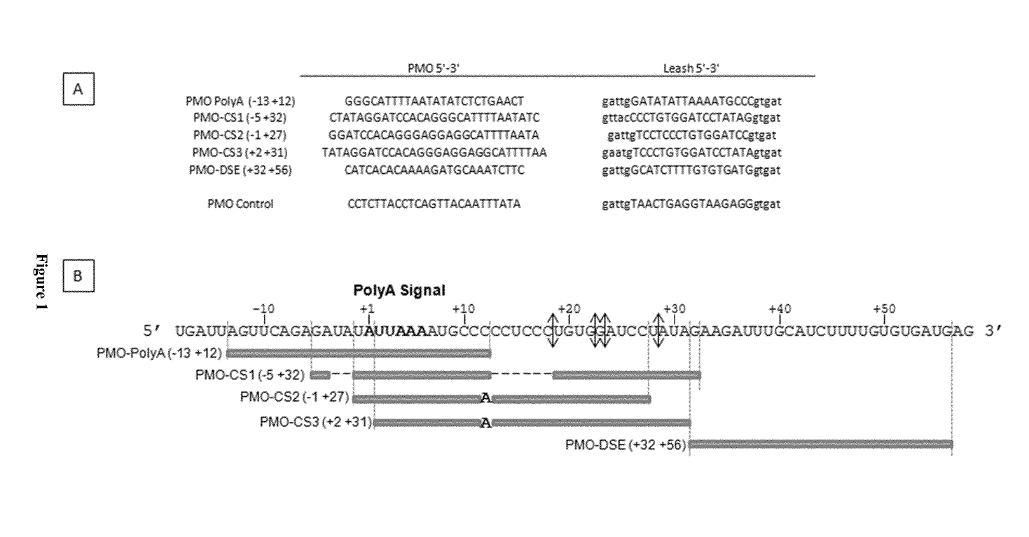

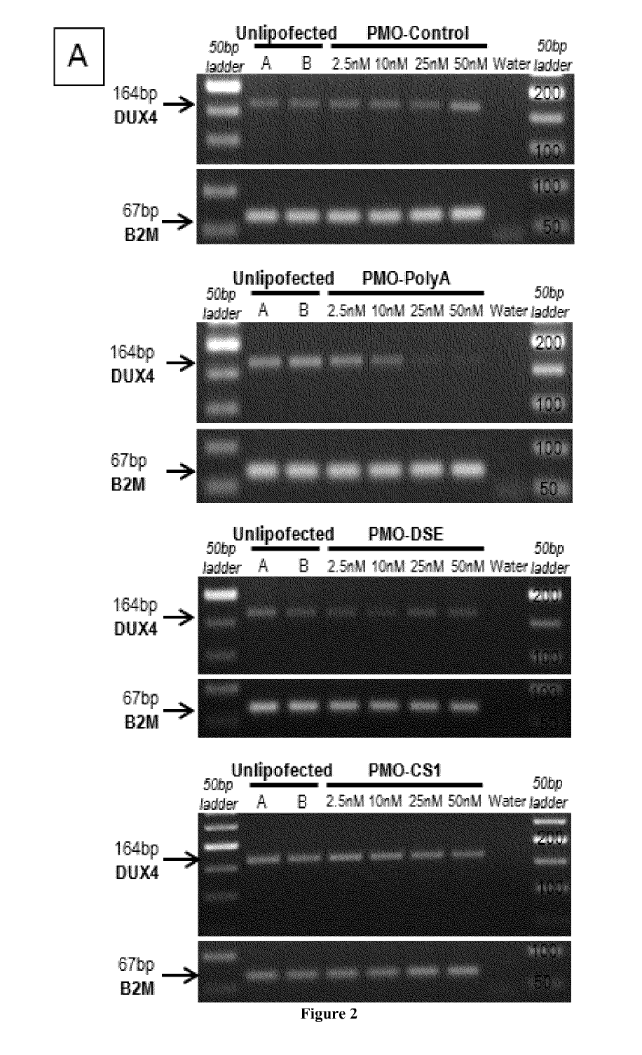

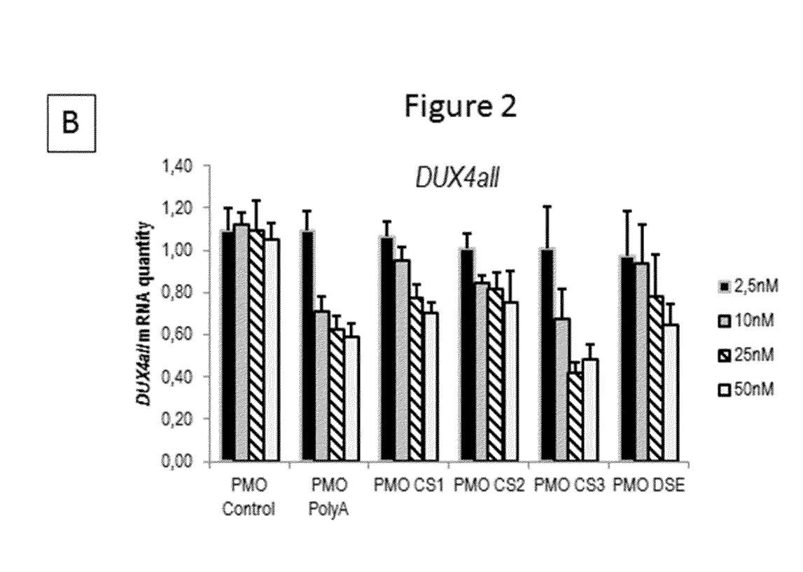

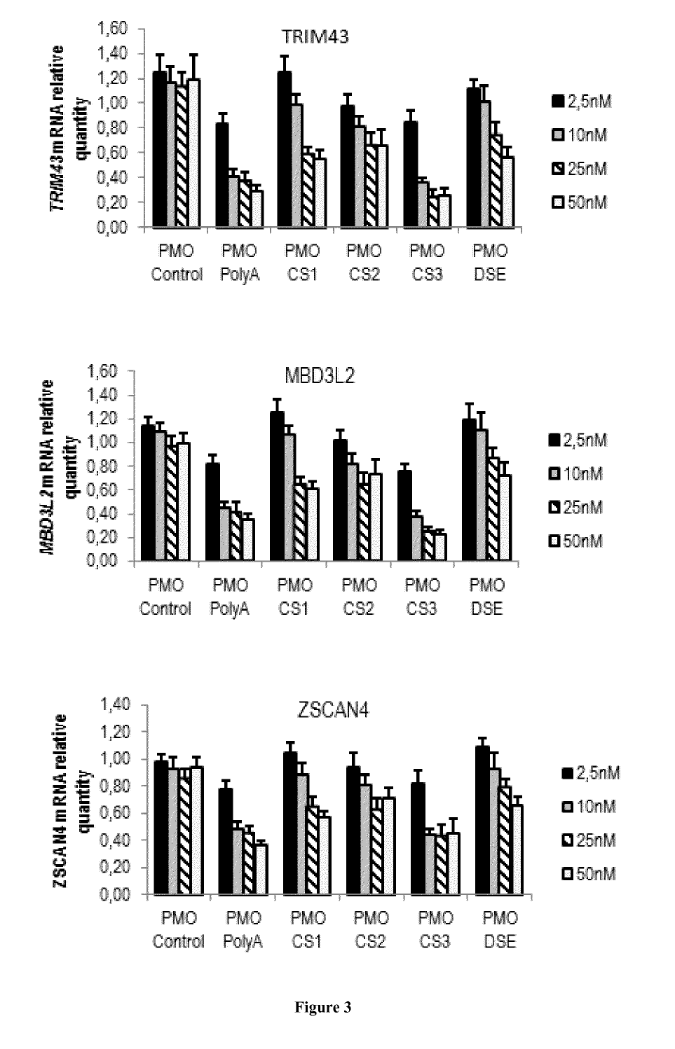

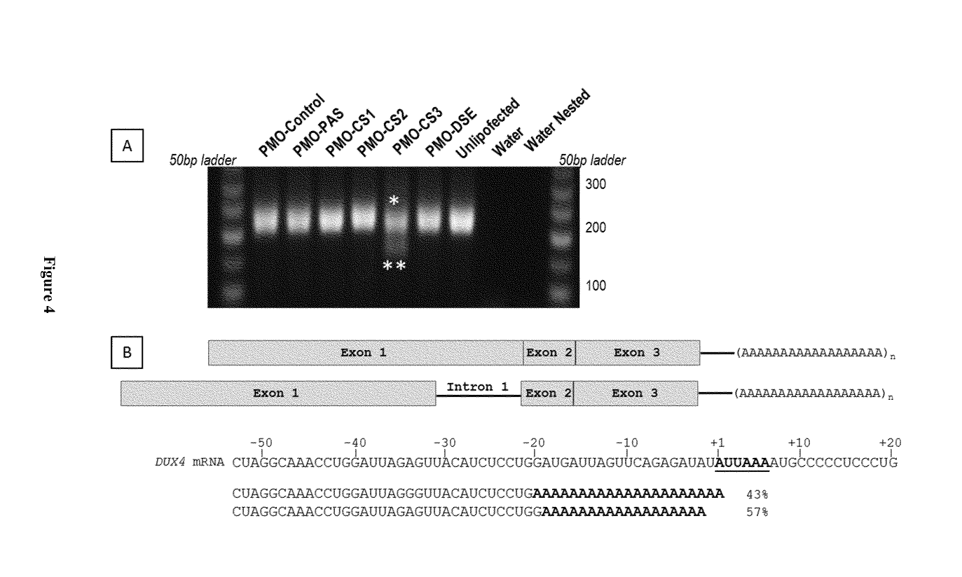

PMO were manufactured and supplied by Gene Tools (LLC, Philomath, USA). The DNA leashes for PMO transfection were synthesized by Eurogentec. The sequences of the PMOs and the leashes are indicated in FIG. 1. The PMOs (2.5 μl at 1 mM) were annealed with the leash (25 μl at 100 μM) in final volume of 50 μL at 95° C. for 5 min, 85° C. for 1 min, 75° C. for 1 min, 65° C. for 5 min, 55° C. for 1 min, 45° C. for 1 min, 35° C. for 5 min, 25° C. for 1 min and then hold at 15° C. Leashed PMOs are stored at −20° C.

Cell Culture and Transfection

Immortalized FSHD cells were cultivated in proliferation medium [4 vols of DMEM (Dulbecco's modified Eagle medium), 1 vol of 199 medium, FBS (Fetal Bovine Serum) 20%, gentamycin 50 μg/mL (Life Technologies, Saint Aubin, France)] supplemented with insulin 5 μg/mL, dexamethasone 0.2 μm/mL, β-FGF 0.5 ng/mL, hEGF 5 ng/ml and fetuine 25 μg/mL. The differentiation was induced by replacing the proliferation medium by DMEM supplemented with insulin (10 μg/mL). Cells were transfected, two days after differentiation induction, with PMO-leashed using Lipofectamine® RNAiMax Reagent (Life Technologies) according to the manufacturer's instructions. Cells were harvested two days after transfection.

RNA Extraction, Reverse Transcription, PCR and Real-Time PCR:

RNA extraction was performed using Trizol according to manufacturer protocol (Life Technologies, Saint Aubin, France). RNA concentration was determined using a nanodrop ND-1000 spectrophotometer (Thermo Scientific, Wilmington, USA). Reverse transcription was done on 1 μg of total RNA (Roche Transcriptor First Strand cDNA Synthesis Kit, Roche, Meylan, France) using oligo GCGAGCTCCGCGGCCGCGTTTTTTTTTTTVN (SEQ ID NO:6). The PCR for DUX4 was performed on 1 μL of RT products using the following program: 94° C. for 5 min, followed by 35 cycles at 94° C. for 20 s and 60° C. for 20 s and 72° C. for 20 s, finished with 72° C. for 7 min. The qPCRs were performed in a final volume of 9 μL with 4 μL of RT product, 0.18 μL of each forward and reverse primers at 20 μM, and 4.5 μL of SYBR® Green MasterMix 2× (Roche, Meylan, France). The qPCR cycling conditions were 94° C. for 5 min, followed by 50 cycles at 94° C. for 10 s and 60° C. for 5 s and 72° C. for 5 s.

| Targeted | | | SEQ | |
|---|---|---|---|---|
| gene | Primer | Sequence | ID NO | Size |
| B2M | B2M_fw | CTCTCTTTCTGG | 7 | 67 bp |
| | | CCTGGAGG | | |
| | B2M_rev | TGCTGGATGACG | 8 | |
| | | TGAGTAAACC | | |
| DUX4-all | DUX4-all_fw | CCCAGGTACCAG | 9 | 164 bp |
| | | CAGACC | | |
| | DUX4-all_rev | TCCAGGAGATGT | 10 | |
| | | AACTCTAATCCA | | |
| MBD3L2 | MBD3L2_fw | CGTTCACCTCTT | 11 | 142 bp |
| | | TTCCAAGC | | |
| | MBD3L2_rev | AGTCTCATGGGG | 12 | |
| | | AGAGCAGA | | |
| ZSCAN4 | VM_ZSCAN4_962U20 | CTGGAGCAGTTT | 13 | 162 bp |
| | | ATGATTGG | | |
| | ZSCAN4_rev | AGCTTCCTGTCC | 14 | |
| | | CTGCATGT | | |
| TRIM 43 | TRIM43_fw | ACCCATCACTGG | 15 | 100 bp |
| | | ACTGGTGT | | |
| | TRIM43_rev | CACATCCTCAAA | 16 | |
| | | GAGCCTGA | | |

Results

Determination of 3′ End Key Elements of DUX4 mRNA and PMO Design

For muscle tissue, one PAS (AUUAAA) has been described for DUX4 mRNA, located 766 bp downstream the stop codon in the 3′UTR (Lemmers et al 2010). We precisely determined the cleavage site of DUX4 by RT-3′RACE-PCR using primers allowing the amplification of all DUX4 isoforms (DUX4-all). Total RNAs were extracted from FSHD myotubes at day 4 of differentiation when DUX4 expression is the highest. The sequence of the amplicon revealed the presence of at least 3 different cleavage sites located 12 to 22 b after the PAS (vertical double arrows in FIG. 1B). In order to investigate the therapeutic potential of AON targeting 3′ key elements of DUX4 mRNA, we designed AO covering either the PAS (PMO-PAS), the cleavage sites (PMO-CS1-3) or the U/GU-rich (PMO-DSE) sequence. The sequences targeted by each PMO are indicated (FIG. 1B).

PMOs Induce a Down Expression of DUX4 mRNA

The efficacy of each PMO was evaluated in a dose dependent manner after transfection in immortalized FSHD clones. Total RNAs were extracted from myotubes and RT-PCR allowing the detection of DUX4-all. No modification of DUX4-all mRNA was observed with PMO-control compared to non-transfected cells thus showing that introduction of PMO-control does not modify DUX4 expression. All the AO designed were efficient in inducing dose-dependent destruction of DUX4-all, although the best efficacies were obtained with the PMO-PAS and -CS3 (FIG. 2B) with 59% and 48% of residual mRNA at 50 nM respectively.

PMOs Induce a Down-Expression of Genes Downstream of DUX4 in FSHD Cells

The expression of 3 genes downstream of DUX4 (TRIM43, MBD3L2 and ZSCAN4) was also investigated by RT-qPCR. All the AO designed were efficient in down regulating genes downstream of DUX, although the best down-regulation was obtained with the PMO-PAS and -CS3. Consistent with the dose-dependent inhibition of DUX4-all expression obtained with these PMOs, the down-regulation of the genes downstream of DUX4 was also dose-dependent (FIG. 3). The percentage of TRIM43 residual mRNAs was 32% and 26% for PMO-PAS and -CS3 respectively at the highest tested dose. For MBD3L2, these percentages were 37% and 22% and for ZSCAN4, the percentages were 39% and 45% for PMO-PAS and -CS3 respectively.

PMO-CS3 Induces a Redirection of Cleavage Region

A redirection of the poly(A) and/or cleavage sites was investigated in the presence of the different PMOs at the highest concentration by 3′RACE nested PCR using forward primers located in Exon 3. A switch in cleavage site or poly(A) usage was not observed with any of the PMOs except PMO-CS3. The sequence of this supplemental band revealed that the cleavage site of the residual DUX4 mRNA in the presence of PMO-CS3 was ˜40 nt upstream of the canonical cleavage site (FIG. 4B), thus suggesting that an alternative PAS was used to generate this new cleavage site

### REFERENCES

Beaudoing, E., S. Freier, et al. (2000). “Patterns of variant polyadenylation signal usage in human genes.” Genome Res 10(7): 1001-10.
Betts, C., Saleh, A. F., Arzumanov, A. A., Hammond, S. M., Godfrey, C., Coursindel, T., Gait, M. J., and Wood, M. J. (2012). Pip6-PMO, A New Generation of Peptide-oligonucleotide Conjugates With Improved Cardiac Exon Skipping Activity for DMD Treatment. Molecular therapy Nucleic acids 1, e38.
Broucqsault, N., J. Morere, et al. (2013). “Dysregulation of 4q35- and muscle-specific genes in fetuses with a short D4Z4 array linked to Facio-Scapulo-Humeral Dystrophy.” Hum Mol Genet 22(20): 4206-4214.
Cazzella, V., Martone, J., Pinnaro, C., Santini, T., Twayana, S. S., Sthandier, O., D'Amico, A., Ricotti, V., Bertini, E., Muntoni, F., et al. (2012). Exon 45 skipping through U1-snRNA antisense molecules recovers the Dys-nNOS pathway and muscle differentiation in human DMD myoblasts. Mol Ther 20, 2134-2142.
Danckwardt, S., M. W. Hentze, et al. (2008). “3′ end mRNA processing: molecular mechanisms and implications for health and disease.” Embo J 27(3): 482-98.
De Angelis, F. G., Sthandier, O., Berarducci, B., Toso, S., Galluzzi, G., Ricci, E., Cossu, G., and Bozzoni, I. (2002). Chimeric snRNA molecules carrying antisense sequences against the splice junctions of exon 51 of the dystrophin pre-mRNA induce exon skipping and restoration of a dystrophin synthesis in Delta 48-50 DMD cells. Proc Natl Acad Sci USA 99, 9456-9461.
Dixit, M., E. Ansseau, et al. (2007). “DUX4, a candidate gene of facioscapulohumeral muscular dystrophy, encodes a transcriptional activator of PITX1.” Proc Natl Acad Sci USA 104(46): 18157-62.
Elkon, R., A. P. Ugalde, et al. (2013). “Alternative cleavage and polyadenylation: extent, regulation and function.” Nat Rev Genet 14(7): 496-506.
Ferreboeuf, M., V. Mariot, et al. (2014). “DUX4 and DUX4 downstream target genes are expressed in fetal FSHD muscles.” Hum Mol Genet 23(1): 171-181.
Gabriels, J., M. C. Beckers, et al. (1999). “Nucleotide sequence of the partially deleted D4Z4 locus in a patient with FSHD identifies a putative gene within each 3.3 kb element.” Gene 236(1): 25-32.
Geng, L. N., Z. Yao, et al. (2012). “DUX4 Activates Germline Genes, Retroelements, and Immune Mediators: Implications for Facioscapulohumeral Dystrophy.” Dev Cell 22: 38-51.
Goyenvalle, A., Vulin, A., Fougerousse, F., Leturcq, F., Kaplan, J. C., Garcia, L., and Danos, O. (2004). Rescue of dystrophic muscle through U7 snRNA-mediated exon skipping. Science 306, 1796-1799.
Jones, T. I., J. C. Chen, et al. (2012). “Facioscapulohumeral muscular dystrophy family studies of DUX4 expression: evidence for disease modifiers and a quantitative model of pathogenesis.” Hum Mol Genet: 4419-30.
Kowaljow, V., A. Marcowycz, et al. (2007). “The DUX4 gene at the FSHD1A locus encodes a pro-apoptotic protein.” Neuromuscul Disord 17(8): 611-23.
Kumar, P., Wu, H., McBride, J. L., Jung, K. E., Kim, M. H., Davidson, B. L., Lee, S. K., Shankar, P., and Manjunath, N. (2007). Transvascular delivery of small interfering RNA to the central nervous system. Nature 448, 39-43.
Lemmers, R. J., P. de Kievit, et al. (2002). “Facioscapulohumeral muscular dystrophy is uniquely associated with one of the two variants of the 4q subtelomere.” Nat Genet 32(2): 235-6.
Lemmers, R. J., J. J. Goeman, et al. (2014). “Inter-individual differences in CpG methylation at D4Z4 correlate with clinical variability in FSHD1 and FSHD2.” Hum Mol Genet.
Lemmers, R. J., R. Tawil, et al. (2012). “Digenic inheritance of an SMCHD1 mutation and an FSHD-permissive D4Z4 allele causes facioscapulohumeral muscular dystrophy type 2.” Nat Genet 44(12): 1370-4.
Lemmers, R. J., P. J. van der Vliet, et al. (2010). “A unifying genetic model for facioscapulohumeral muscular dystrophy.” Science 329(5999): 1650-3.
Lundberg, M., Wikstrom, S., and Johansson, M. (2003). Cell surface adherence and endocytosis of protein transduction domains. Mol Ther 8, 143-150.
Malhotra, M., Tomaro-Duchesneau, C., Saha, S., Kahouli, I., and Prakash, S. (2013). Development and characterization of chitosan-PEG-TAT nanoparticles for the intracellular delivery of siRNA. International journal of nanomedicine 8, 2041-2052.
McCarty, D. M., Monahan, P. E., and Samulski, R. J. (2001). Self-complementary recombinant adeno-associated virus (scAAV) vectors promote efficient transduction independently of DNA synthesis. Gene Ther 8, 1248-1254.
Mitsuhashi, H., S. Mitsuhashi, et al. (2013). “Expression of DUX4 in zebrafish development recapitulates facioscapulohumeral muscular dystrophy.” Hum Mol Genet 22(3): 568-77.
Nunes, N. M., W. Li, et al. (2010). “A functional human Poly(A) site requires only a potent DSE and an A-rich upstream sequence.” Embo J 29(9): 1523-36.
Sachs, A. (1990). “The role of poly(A) in the translation and stability of mRNA.” Curr Opin Cell Biol 2(6): 1092-8.
Schumperli, D., and Pillai, R. S. (2004). The special Sm core structure of the U7 snRNP: far-reaching significance of a small nuclear ribonucleoprotein. Cell Mol Life Sci 61, 2560-2570.
Snider, L., A. Asawachaicharn, et al. (2009). “RNA transcripts, miRNA-sized fragments and proteins produced from D4Z4 units: new candidates for the pathophysiology of facioscapulohumeral dystrophy.” Hum Mol Genet 18(13): 2414-30.
Snider, L., L. N. Geng, et al. (2010). “Facioscapulohumeral dystrophy: incomplete suppression of a retrotransposed gene.” PLoS Genet 6(10): e1001181.
Tawil, R., S. M. van der Maarel, et al. (2014). “Facioscapulohumeral dystrophy: the path to consensus on pathophysiology.” Skelet Muscle 4: 12.
Thomas, N. S., K. Wiseman, et al. (2007). “A large patient study confirming that facioscapulohumeral muscular dystrophy (FSHD) disease expression is almost exclusively associated with an FSHD locus located on a 4qA-defined 4qter subtelomere.” J Med Genet 44(3): 215-8.
Tian, B., J. Hu, et al. (2005). “A large-scale analysis of mRNA polyadenylation of human and mouse genes.” Nucleic Acids Res 33(1): 201-12.
Tupler, R., A. Berardinelli, et al. (1996). “Monosomy of distal 4q does not cause facioscapulohumeral muscular dystrophy.” J Med Genet 33(5): 366-70.
van der Maarel, S. M., D. G. Miller, et al. (2012). “Facioscapulohumeral muscular dystrophy: consequences of chromatin relaxation.” Curr Opin Neurol 25(5): 614-20.
van Deutekom, J. C., C. Wijmenga, et al. (1993). “FSHD associated DNA rearrangements are due to deletions of integral copies of a 3.2 kb tandemly repeated unit.” Hum Mol Genet 2(12): 2037-42.
Vanderplanck, C., E. Ansseau, et al. (2011). “The FSHD Atrophic Myotube Phenotype Is Caused by DUX4 Expression.” PLoS One 6(10): e26820.
Wallace, L. M., S. E. Garwick, et al. (2011). “DUX4, a candidate gene for facioscapulohumeral muscular dystrophy, causes p53-dependent myopathy in vivo.” Ann Neurol.
Wallace, L. M., J. Liu, et al. (2012). “RNA Interference Inhibits DUX4-induced Muscle Toxicity In Vivo: Implications for a Targeted FSHD Therapy.” Mol Ther.
Wijmenga, C., J. E. Hewitt, et al. (1992). “Chromosome 4q DNA rearrangements associated with facioscapulohumeral muscular dystrophy.” Nat Genet 2(1): 26-30.
Xu, H., Z. Wang, et al. (2014). “Dux4 induces cell cycle arrest at G1 phase through upregulation of p21 expression.” Biochem Biophys Res Commun 446(1): 235-40.
Yao, Z., L. Snider, et al. (2014). “DUX4-induced gene expression is the major molecular signature in FSHD skeletal muscle.” Hum Mol Genet.
Yamada, T., Das Gupta, T. K., and Beattie, C. W. (2013). p28, an anionic cell-penetrating peptide, increases the activity of wild type and mutated p53 without altering its conformation. Molecular pharmaceutics 10, 3375-3383.

1. An antisense oligonucleotide that hybridizes with one or more key elements of the polyadenylation region of a target pre-mRNA, wherein said key element(s) is selected in the group consisting of cleavage site(s) and the U:GU-rich region (or DSE for DownStream Element) of said pre-mRNA.

2. The antisense oligonucleotide according to claim 1, comprising from about 10 to about 40 nucleotides.

3. The antisense oligonucleotide according to claim 1, which is a PMO, 2′-O-methyl, tricyclo-DNA or tricyclo-phosphorothioate DNA oligonucleotide.

4. The antisense oligonucleotide according to claim 1, which is annealed to a sense oligonucleotide, said sense oligonucleotide optionally comprising nucleotides that protrudes from one or both of 5′ and 3′ ends of the antisense oligonucleotides.

5. The antisense oligonucleotide according to claim 1, wherein the target pre-mRNA is a DUX4 pre-mRNA.

6. The antisense oligonucleotide according to claim 5, wherein said antisense oligonucleotide is selected in the group consisting of SEQ ID NO:2 to 5.

7. A vector for delivering the antisense oligonucleotide according to claim 1.

8. The vector according to claim 7, which is a viral vector coding said antisense oligonucleotide.

9. A composition comprising an antisense oligonucleotide according claim 1 or a vector according to claim 1.

10. The composition according to claim 7, comprising an antisense oligonucleotide and a nucleic acid transfection reagent such as a cationic lipid.

11. The antisense oligonucleotide according to claim 1, for use in a method for the treatment of a disease mediated by said pre-mRNA or by a protein encoded by said pre-mRNA.

12. The antisense oligonucleotide for use according to claim 11, wherein the pre-mRNA is a DUX4 pre-mRNA and the disease is Facioscapulohumeral dystrophy.

13. The vector according to claim 7, for use in a method for the treatment of a disease mediated by said pre-mRNA or by a protein encoded by said pre-mRNA.

14. The composition according to claim 9, for use in a method for the treatment of a disease mediated by said pre-mRNA or by a protein encoded by said pre-mRNA.

15. The vector for use according to claim 13, wherein the pre-mRNA is a DUX4 pre-mRNA and the disease is Facioscapulohumeral dystrophy.

16. The composition for use according to claim 14, wherein the pre-mRNA is a DUX4 pre-mRNA and the disease is Facioscapulohumeral dystrophy.


SEQUENCE LISTING (27 sequences)

SEQ ID NO: 1 - DNA, 25 bp, artificial
  poly A sequence AO
gggcattttaatatatctctgaact

SEQ ID NO: 2 - DNA, 29 bp, artificial
  CS1 AO
ctataggatccacagggcattttaatatc

SEQ ID NO: 3 - DNA, 28 bp, artificial
  CS2 AO
ggatccacagggaggaggcattttaata

SEQ ID NO: 4 - DNA, 30 bp, artificial
  CS3 AO
tataggatccacagggaggaggcattttaa

SEQ ID NO: 5 - DNA, 25 bp, artificial
  DSE AO
catcacacaaaagatgcaaatcttc

SEQ ID NO: 6 - DNA, 31 bp, artificial
  poly dToligonucleotide
  misc_feature (31)..(31): n is a, c, g, or t
gcgagctccgcggccgcgtttttttttttvn

SEQ ID NO: 7 - DNA, 20 bp, artificial
  primer
ctctctttctggcctggagg

SEQ ID NO: 8 - DNA, 22 bp, artificial
  primer
tgctggatgacgtgagtaaacc

SEQ ID NO: 9 - DNA, 18 bp, artificial
  primer
cccaggtaccagcagacc

SEQ ID NO: 10 - DNA, 24 bp, artificial
  primer
tccaggagatgtaactctaatcca

SEQ ID NO: 11 - DNA, 20 bp, artificial
  primer
cgttcacctcttttccaagc

SEQ ID NO: 12 - DNA, 20 bp, artificial
  primer
agtctcatggggagagcaga

SEQ ID NO: 13 - DNA, 20 bp, artificial
  primer
ctggagcagtttatgattgg

SEQ ID NO: 14 - DNA, 20 bp, artificial
  primer
agcttcctgtccctgcatgt

SEQ ID NO: 15 - DNA, 20 bp, artificial
  primer
acccatcactggactggtgt

SEQ ID NO: 16 - DNA, 20 bp, artificial
  primer
cacatcctcaaagagcctga

SEQ ID NO: 17 - DNA, 25 bp, artificial
  Control
cctcttacctcagttacaatttata

SEQ ID NO: 18 - DNA, 27 bp, artificial
  polyA leash
gattggatatattaaaatgcccgtgat

SEQ ID NO: 19 - DNA, 27 bp, artificial
  CS1 leash
gttacccctgtggatcctataggtgat

SEQ ID NO: 20 - DNA, 26 bp, artificial
  CS2 leash
gattgtcctccctgtggatccgtgat

SEQ ID NO: 21 - DNA, 27 bp, artificial
  CS3 leash
gaatgtccctgtggatcctatagtgat

SEQ ID NO: 22 - DNA, 27 bp, artificial
  DSE leash
gattggcatcttttgtgtgatggtgat

SEQ ID NO: 23 - DNA, 26 bp, artificial
  control leash
gattgtaactgaggtaagagggtgat

SEQ ID NO: 24 - RNA, 76 nt, homo sapiens
ugauuaguucagagauauauuaaaaugcccccucccuguggauccuauagaagauuugcaucuuuugugugaugag

SEQ ID NO: 25 - RNA, 73 nt, Homo sapiens
cuaggcaaaccuggauuagaguuacaucuccuggaugauuaguucagagauauauuaaaaugcccccucccug

SEQ ID NO: 26 - RNA, 54 nt, Homo sapiens
cuaggcaaaccuggauuaggguuacaucuccugaaaaaaaaaaaaaaaaaaaaa

SEQ ID NO: 27 - RNA, 52 nt, Homo sapiens
cuaggcaaaccuggauuagaguuacaucuccuggaaaaaaaaaaaaaaaaaa